(19) United States
(12) Plant Patent Application Publication
(10) Pub. No.: US 2026/0114340 P1
(43) Pub. Date: Apr. 23, 2026

(54) Black Locust Tree Named 'OBE TSZM'

(50) ;

(71) Applicant: Hungaroplant Kft, Budapest (HU)

(72) Inventor: Jeno Nemeth, Budapest (HU)

(21) Appl. No.: 19/363,142

(22) Filed: Oct. 20, 2025

Related U.S. Application Data
(60) Provisional application No. 63/709,692, filed on Oct. 21, 2024.

(51) Int. Cl. A01H 6/00; A01H 5/00

(52) U.S. Cl. CPC A01H 6/00; A01H 5/00
USPC PLT/216

(57) ABSTRACT

A new black locust tree ‘OBE TSZM’ particularly distinguished by elliptic leaves with an obtuse apex; bodkin like thorns; straight, pinnate growth habit; and blooms profusely and is a useful bee pasture, well-liked by beekeepers, is disclosed.

### CROSS REFERENCE TO RELATED MATTER

The present application claims priority to U.S. Application No. 63/709,692, as filed on Oct. 21, 2024, the entire contents of which is incorporated herein by reference for all purposes.

Genus and species: Robinia pseudoacacia.

Variety denomination: ‘OBE TSZM’.

### BACKGROUND OF THE NEW PLANT ‘OBE TSZM’

The present invention comprises a new and distinct variety of black locust tree, botanically known as Robinia pseudoacacia and hereinafter referred to by the variety name ‘OBE TSZM’. ‘OBE TSZM’ was selected from experimental Black locust (Robinia pseudoacacia) plantation established on the Great Hungarian Plain near town Nagykőrös, in 2020.

‘OBE TSZM’ was asexually reproduced in 2020 by vegetative cuttings from budwood.

### SUMMARY OF ‘OBE TSZM’

The following are the most outstanding and distinguishing characteristics of this new variety when grown under normal horticultural practices near Nagykőrös, Hungary.
1. Elliptic leaves with an obtuse apex;
2. Bodkin like thorns;
3. Straight, pinnate growth habit; and
4. Blooms profusely and is a useful bee pasture, well-liked by beekeepers.

### DESCRIPTION OF THE PHOTOGRAPHS

This new Black Locust tree is illustrated by the accompanying photographs of plants grown near Nagykőrös, Hungary. The colors shown are as true as can be reasonably obtained by conventional photographic procedures.

FIG. 1 shows the overall shape of the new ‘OBE TSZM’ variety.

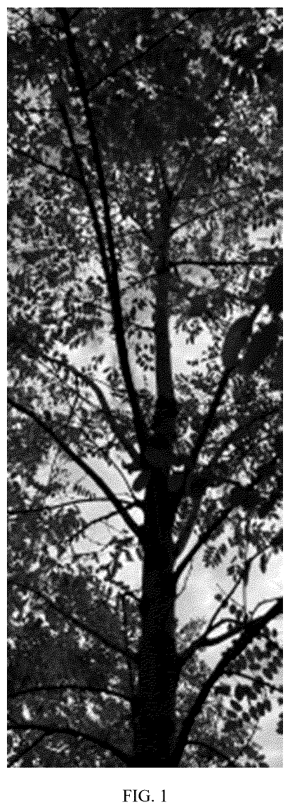

FIG. 2 shows a close-up of the bark and thorns of ‘OBE TSZM’.

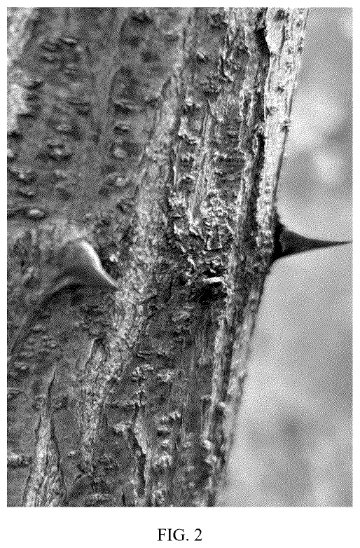

FIG. 3 shows a close-up view of the leaves of ‘OBE TSZM’.

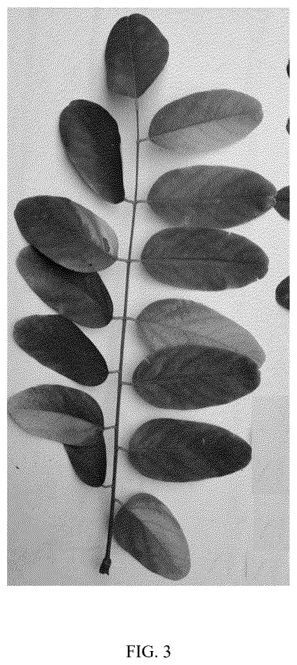

### DESCRIPTION OF THE ‘OBE TSZM’ VARIETY

The following detailed descriptions set forth the distinctive characteristics of ‘OBE TSZM’. The data which define these characteristics was collected near Nagykőrös, Hungary. Color references are to The R.H.S. Colour Chart of The Royal Horticultural Society of London (R.H.S.), 6th edition (2016).
Classification:
   Family.—Fabaceae.
   Botanical.—Robinia pseudoacacia.
   Common name.—Black locust.
   Variety name.—‘OBE TSZM’.
Parentage:
   Female parent.—Unnamed Experimental black locust.
   Male parent.—Unknown.
General Description:
   Plant habit.—Deciduous tree with straight, upright growth habit. At the age of three, it is a small tree with a diameter of 3.0 to 7.0 cm and a height of 5.0 to 8.0 m.
   Bark color (mature stems).—Predominately Greyed-Brown Group 199B; ridges and older, shaded fissures approach Greyed-Brown Group 199A to Black Group 202A; freshly exfoliated/abraded areas show warm tones near Greyed-Orange Group 165B-165C.
   Crotch angles.—40-50, sometimes with a break upwards of another 50-60.
   Smaller branches in 1.0 m.—Average of 8.33.
   Larger branches in 1.0 m.—Average of 1.33.
   Stem straightness.—No curves, straight stem.
   USDA hardiness zone.—Zone 4.
   Thorns.—Bodkin like.
   Diseases and pest resistance.—Resistant to higher percentages of calcium carbonate in the soil, and higher soil pH.
Foliage description:
   Color.—Medium green.
   Variegation.—Absent.
   Leaf shape.—Middle section of leaf: Elliptic. Apex: Obtuse.
Flowers description:
   Conspicuousness.—Medium.
   Type.—Single.
   Color.—White.
Fruit: Absent.

I claim:

1. A new and distinct variety of Black Locust tree named ‘OBE TSZM’ as shown and described herein.
